(19) United States
(12) Patent Application Publication
(10) Pub. No.: US 2019/0247429 A1
(43) Pub. Date: Aug. 15, 2019

(54) Use of nano-material composition DG-5 for the treatment of drug-resistant bacteria

(71) Applicant: CHANGSHA DIGU NANO BIOTECHNOLOGY CO., LTD., Changsha (CN)

(72) Inventors: Jinjun Liu, Changsha (CN); Qiangbai B.H. Li, Changsha (CN); Jianchao Situ, Changsha (CN)

(73) Assignee: CHANGSHA DIGU NANO BIOTECHNOLOGY CO., LTD., Changsha (CN)

(21) Appl. No.: 16/350,779

(22) PCT Filed: Jul. 11, 2017
(86) PCT No.: PCT/CN2017/000429

§ 371 Date: Jan. 11, 2019

(30) Foreign Application Priority Data
Jul. 13, 2016 (CN) 201610548369.5

(51) Int. Cl. A61K 33/38; A61P 31/04; A61K 9/16

(52) U.S. Cl. CPC A61K 33/38; A61K 9/16; A61P 31/04

(57) ABSTRACT

The present invention is in the field of medicinal technologies, and provides a nano-material composition DG-5 for use as a medicament against drug-resistant bacteria. The composition showed a strong inhibitory effect against several super drug-resistant bacteria (superbugs), and thus can be used for manufacture of novel effective antibacterial drugs. The mediocament can be administered externally, orally, subcutaneously, or via intravenous or intramuscular injection.

### FIELD OF THE INVENTION

The present invention, as in the field of medical technologies, relates to a nano-material composition DG-5 for use as a medicament against drug-resistant bacteria. The composition showed a strong inhibitory effect against drug-resistant bacteria, such as methicillin-resistant Staphylococcus aureus (MRSA) and vancomycin-resistant Enterococci (VRE), and especially against super drug-resistant bacteria (superbugs), such as Enterobacter cloacae, Pseudomonas aeruginosa, Escherichia coli, Klebsiella pneumoniae, and Acinetobacter baumannii.

### BACKGROUND OF THE INVENTION

In recent years, rising drug resistance has posed a seriously threat to health and life in the public. As early as the 1940s, penicillin, as the first effective antibiotic, successfully solved the problem of clinical staphylococcus aureus infection. Subsequently developed antibiotics, such as macrolides and aminoguanidine antibiotics, have greatly reduced the mortality rate of deadly diseases such as pneumonia and tuberculosis. However, the era of human victory over bacteria is far from coming. In fact, many antibiotics have experienced varying degrees of efficacy reduction after years of use. Natural penicillins have long lost their medicinal value in controlling the infection of staphylococcus aureus. In the 1950s and 1960s of the “golden age” of antibiotics, the number of people dying from infectious diseases worldwide was about 7 millions, and this number rose to more than 20 millions in 1999. In the United States, the world's most technologically advanced country, the number of people dying from infectious diseases increased by 40% between 1982 and 1992, and the number of people dying from sepsis rose by 89%. The main cause of the increase in mortality is the difficulty of medications caused by drug-resistant bacteria.

For a long time, bacterial drug-resistance has not received enough attention, and some physicians believed that existing drugs are sufficient to treat drug-resistant bacteria. For example, ampicillin and cephalosporin were used against Staphylococcus aureus which is resistant to natural penicillin, or even vancomycin can be used against methicillin-resistant Staphylococcus aureus (MRSA) which is resistant to cephalosporin. In 1992, however, vancomycin-resistant MRSA was first discovered in the United States. The US Centers for Disease Control and Prevention confirmed in May 2016 that the first bacteria carrying the MCR-1 gene were found as “invincible bacteria” cases; the bacteria were found resistant to all antibiotics at this stage, including colistin which was used as the last ditch of defense but was stopped using in human in the 1970s and 1980s due to its damage to human kidneys. This is an alarm call for human abuse of antibiotics.

Abuse of antibiotics is found very common in China, where antibiotics has been used much often than some other countries. At the same time, physicians have abused broad-spectrum antibacterial drugs such as ampicillin, which leads to the imbalance of flora in the human body and induces secondary infections. Recently Chinese and British researchers reported in the journal of Lancet Infectious Diseases that the bacteria carrying MCR-1 gene have been found in poultry and humans in China without much attention in the public. Up to now, drug-resistance of bacteria has become a very prominent issue in China. The number of hospital-infected patients caused by drug-resistant bacteria has accounted for about 30% of the total number of hospitalized patients. How to solve the problem of bacterial resistance is already urgent. In addition to enhancing the training for medical practitioners, strictly eliminating the abuse of antibiotics and establishing dynamic monitoring of bacterial resistance, it is imperative to catch up in the development of new antibiotics.

With the increasing resistance of drug-resistant bacteria, and even the emergence of super drug-resistant bacteria or super drug-resistant bacteria (“superbugs”), the world's major pharmaceutical companies recently have begun to strengthen investment in the development of new antibiotics. It would be of great significance to develop novel drugs with novel structure and low toxicity against drug-resistant bacteria. In recent years, there are also a small number of patents issued in China based on researches of drug-resistant bacteria: CN101584694A, CN101195627A, CN101428026A, CN100586433C, CN100441580C, CN1308047A, CN101074235A, CN100519533C, CN101786979A, and CN102464603B. Among them, CN102464603B provided an indandione derivative and use thereof in preparation of a medicament against drug-resistant bacteria with a strong inhibitory effect to especially methicillin-resistant staphylococcus aureus and other drug-resistant bacteria.

On the other hand, metallic silver has reported to have a broad-spectrum bactericidal effect. Silver ions and silver-containing compounds may be used to kill or inhibit bacteria, viruses, algae and fungi. Silver is considered to be a biotropic metal due to its effect in the treatment of diseases. Silver is harmless to normal human cells. The antibacterial properties of silver have been widely used in the pharmaceutical industry since the 16th century. The Compendium of Materia Medica written by Li Shizhen, a famous Chinese medicine practitioner of the Ming Dynasty, also reported silver's antibacterial uses, such as covering wound to prevent ulceration, wrapping wounded skin with gauze with silver cloth, applying a silver nitrate solution on a new born infant to prevent mucosal infection. In the 1930s, the discovery of antibiotics once led to the neglect of the utility of silver antibacterial properties. However, with the abuse of chemical drugs such as antibiotics, more and more microbes have developed drug resistance through mutations, making some diseases caused by drug-resistant bacteria incurable. In recent years, metallic silver attracted attention as a metallic material with high bio-safety and stability, and also attracted attention to silver germicidal agents with high efficiency, broad spectrum, and low tendency to develop drug resistance.

As the metallic silver has broad-spectrum, long-acting and strong antibacterial effects, has no resistance to drugs and irritating reaction to the skin or any toxic reaction, can promote wound healing, cell growth and damaged cells, silver-containing medical devices have attracted attention in recent years. Nano-silver antibacterial fibers, nano-silver wound dressings, nano-silver gels, nano-silver antibacterial catheters, and nano-silver condoms have been designed and developed. Since 2004, there have been 29 kinds of medical products containing nano-silver approved by the provincial Food and Drug Administration and entered clinical application. In February 2016, the China National Food and Drug Administration approved the first seven products to enter clinical applications.

However, most of the nano-silver powders on the market are prepared by chemical methods, wherein various particle sizes and shapes are mixed. Thus, it is difficult to determine and ensure to have appropriate purity, distribution, performance and stability. Further researches need to be carried out on the technical issues such as of collection, storage, transportation of the nano-silver powders, and the safety issue is of much concern for biological and pharmaceutical applications. Most of nano-silver products on the market, as restricted by the existing technology of preparation, are made from elemental metallic silver and silver-containing compounds by using physical and chemical methods. Conventional physical methods, such as ball milling, in the preparation process are difficult to reach nano scale. Electrolytic method has low yield and high cost, and thus is not suitable for industrial use. Chemical methods have always been difficult in obtaining high-purity nano-powder products, especially in industrial processes, it would be extremely difficult to separate various “acid radicals” and remove impurities, and further, reducing agents may also be difficult to be dealt with. Therefore, the use of nano-silver materials were restricted in especially in the fields of biology and medicine, and so far there is no report on the use of nano-silver against super drug-resistant bacteria.

### SUMMARY OF THE INVENTION

The object of the present invention is to provide a nano-material composition DG-5 and use thereof as a medicament against drug-resistant bacteria. The composition showed a strong inhibitory effect against drug-resistant bacteria, such as methicillin-resistant Staphylococcus aureus (MRSA) and vancomycin-resistant Enterococci (VRE), and especially against super drug-resistant bacteria (superbugs), such as Enterobacter cloacae, Pseudomonas aeruginosa, Escherichia coli, Klebsiella pneumoniae, and Acinetobacter baumannii.

The present invention provided a nano-material composition DG-5 for medical use to treat drug-resistant bacteria, including use in manufacture of a medication.

The present invention provided a use of the nano-material composition DG-5 in the manufacture of a medicament, which is used against drug-resistant bacteria, wherein the composition contains: 1-2 g/L of spherical nano-silver powder, 1-2 g/L of glucose, and water as the rest. The spherical nano-silver powder has a particle size of 0.1-5 nm (purchased from Hunan Optics Valley Nano Technology Co., Ltd.), and a purity of silver is ≥99.99%.

The drug-resistant bacteria include Klebsiella pneumoniae, Acinetobacter calcoaceticus, Enterococcus faecalis, Streptococcus pneumoniae or Staphylococcus aureus.

The drug-resistant bacteria may also include super drug-resistant bacteria such as Enterobacter cloacae, Klebsiella pneumoniae, Escherichia coli, Pseudomonas aeruginosa or Acinetobacter baumannii.

In various embodiments, the nano-material composition may be used alone or in combination with other agents.

The present invention provides also an antibacterial agent, which comprises the nano-material composition and one or more pharmaceutically acceptable carriers.

In various embodiments, the carrier is, for example, a diluent, excipient, filler, binder, wetting agent, disintegrant, absorption enhancer, surfactant, adsorption carrier, lubricant, or a combination thereof.

The antibacterial agent, as needed for a patient to treat drug-resistant bacteria, can be in the form of, for example, injection, tablet, pill, capsule, suspension or emulsion.

The inventors conducted extensive researches on the nano-materials by using nanotechnologies. The nano-materials exhibit many special physical and chemical properties because of their unique surface effects, small-size effects, quantum-size effects and macroscopic quantum tunneling effects, which are significantly different from those of the bulk materials of the same substance especially in terms of mechanics, thermals, magnetism, optoelectronics, and electronics. The inventors, based on the research, believe that the development of nanotechnology provides a new direction in anti-infection research, and many nano-materials exhibit potential antibacterial activity. When the metallic silver is processed into nano-silver, the specific surface area becomes extremely large, which shows significant surface effects, small-size effects and macroscopic tunneling effects. These effects in combination greatly enhance the antibacterial ability of silver, especially for ultrafine nano-silver (with the particle size less than 5 nm), so that the effective concentration of antibacterial nano-silver can reach nano-molar level, much lower than the micro-molar level of silver ions.

The present invention employed cutting edge nanotechnology to make silver into nano scale (0.1-100 nm) in size of the particles. Powerful bactericidal effects may be generated with a very small amount of nano-silver, killing more than 650 kinds of bacteria in a matter of minutes. With regard to the nano-silver and the method of preparation, reference can be made to the Chinese patent application CN 201510066287.2, filed Jan. 10, 2015, the disclosure of which is hereby incorporated in its entirety by reference.

The present invention relates to a nano-silver composition DG-5, wherein the nano-silver powder (0.1-5 nm) is obtained from the Hunan Optics Nanotechnology Co., Ltd., and medicinally acceptable glucose and purified water is used as its stabilizer and diluent. The kinds of components are reduced as much as possible, and further, the safety of the composition as a medicament is ensured in terms of the quality of the materials.

### BRIEF DESCRIPTION OF THE DRAWINGS

FIG. 1 illustrates a map of the test panel, wherein, rows A and B: ciprofloxacin (CIP), the highest test concentration being 64 μg/ml, and diluted by 2 times; rows C and D: DG-5, the highest test concentration being 30 μg/ml, and doubling diluted; growth control (GC): compound solvent, containing bacteria with inoculum of 1.1×CAMHB or CAMHBII, no compound being present. Sterile control (SC): compound solvent, 1.1×CAMHB or CAMHBII, no compound being present.

### DETAILED DESCRIPTION OF THE EMBODIMENTS

The invention of this application can be further described by the following examples, but the examples are merely illustrative, and are not intended to limit the scope of the invention.

### Example 1

A nano-material composition DG-5 has the following components: 1-2 g/L of spherical nano-silver powder, 1-2 g/L of glucose, and water as the rest. The spherical nano-silver powder has a particle size of 0.1-5 nm (purchased from Hunan Optics Valley Nano Technology Co., Ltd.), and a purity of silver of ≥99.99%.

### Example 2: Determination of the Activity of the Medicinal Nano-Material Composition DG-5 Against 5 Drug-Resistant Strains: Klebsiella pneumoniae, Acinetobacter Calcoaceticus, Enterococcus Faecalis, Streptococcus Pneumoniae, and Staphylococcus aureus

In this study, the minimum inhibitory concentration (MIC) is used as an indicator of antibacterial activity. MIC refers to the minimum inhibitory concentration of a compound that inhibits the apparent growth of a certain microorganism. MIC is determined by using micro-culture dilution method of the Guidelines of the Clinical and Laboratory Standards Institute (CLSI). In this study, the MICs of one test sample DG-5 and one control antibiotic ciprofloxacin against five strains of bacteria were determined. The test sample DG-5 was doubling-diluted in a 96-well plate from the highest detection concentration of 30 μg/ml. After the test plate was placed in a common incubator at 35±2° C. for 16-20 hours, the bacterial growth in the wells was observed and recorded. The MIC of the reference of ciprofloxacin was found consistent with the historical data, and the MIC of the test sample DG-5 against 5 strains of bacteria was determined to be between 1.875-15 μg/ml.

1. Material

Bacterial Strains

| | Gram staining | | |
|---|---|---|---|
| Bacteria | classification | Serial number | Resistance |
| Klebsiella | G− | ATCC 700603 | AMP, AZT, CFX, |
| pneumoniae | | | CPD, CAZ, CHL, |
| | | | PIP, TET |
| Acinetobacter | G− | ATCC 51432 | IMI |
| calcoaceticus | | | |
| Enterococcus | G+ | ATCC 700221 | VAN |
| faecalis | | | |
| Streptococcus | G+ | ATCC 49619 | PEN |
| pneumoniae | | | |
| Staphylococcus | G+ | ATCC 43300 | MET, OXA |
| aureus | | | |

Medium:

Trypticase soy agar (TSA) (BD BBL 211043) TSA+5% sheep blood (TSA II); cation-adjusted Mueller Hinton broth (CAMHB) (BD BBL212322); CAMHB+3% horse blood (CAMHB II); sheep blood (Quad Five 630-500); horse blood (Quad Five 205-500).

Reagents and Consumables:

The test sample DG-5 (300 μg/ml) was supplied by Changsha Digu Nami. Ciprofloxacin (Sigma 17850). Disposable shake flask, 250 ml (Corning 430183). Disposable plate, 100 mm (VWR 25384-302), and 96-well microtiter plate (Greiner 650162).

2. Methods

Bacterial Resuscitation:

Five strains of bacteria used for the minimum inhibitory concentration test were frozen at −80° C. in a low-temperature refrigerator and resuscitated 2 days earlier. A small amount of frozen bacteria was scrapped with a sterile inoculating loop, streak-inoculated in appropriate solid medium plates, and placed in a suitable gas culture environment at 35±2° C. for 35-24 hours (Streptococcus pneumoniae: TSA II, 5% CO₂; Enterococcus faecalis: TSA II, normal atmospheric environment, the remaining 3 strains of bacteria: TSA, normal atmospheric environment). 5-10 morphologically similar colonies were picked from the above culture dishes using a sterile inoculating loop, re-streaked onto a suitable solid medium plate, and was then placed in a suitable gas culture environment at 35±2° C. for 35-24 hours.

Inoculation of Bacteria:

A liquid medium was taken out of the 4° C. refrigerator and warmed up at room temperature. Five to 10 bacterial single colonies were picked from the above solid culture dish and re-suspended in 500 μl of 1.1×CAMHB, and the OD₆₀₀ was adjusted to 0.1 to 0.13 with a spectrophotometer. Gram-positive bacteria (G+) were diluted 280-fold with the corresponding liquid medium (CAMHB or CAMHBII), and Gram-negative bacteria (G-) were diluted 400-fold (for example, 35.6 μl of G+ bacterial culture diluted in 10 ml of CAMHB or 25 μl of G-bacteria culture diluted in 10 ml of CAMHB).
Streptococcus pneumoniae: CAMHBII
The remaining 4 strains of bacteria: CAMHB

Preparation of Test Plate:

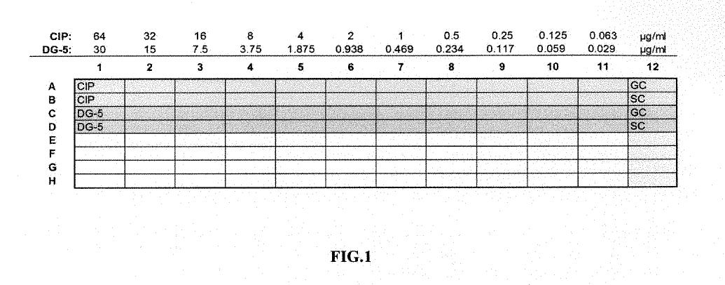

Test plate map (see FIG. 1): Rows A and B: ciprofloxacin (CIP), the highest test concentration being 64 μg/ml, and doubling diluted; rows C and D: DG-5, the highest test concentration being 30 μg/ml, and doubling-diluted; growth control (GC): compound solvent, containing bacteria with inoculum of 1.1×CAMHB or CAMHBII, no compound being present. Sterile control (SC): compound solvent, 1.1×CAMHB or CAMHBII, no compound being present.

Dilution of the Compounds:

To the starting well of the dilution plate was transferred 120 μl of the compound, and to other wells was transferred 60 μl of dimethyl sulfoxide (DMSO for diluting ciprofloxacin) or DG-5. Each compound was serially doubling-diluted from column 1 to column 11 (i.e., 60 μl was taken from column 1 to column 2 and mixed, and 60 μl was taken from column 2 to column 3 and mixed, and then 60 μl was taken from column 3 to column 4 and mixed, and so on, until the column 11). To the corresponding wells of each of the 5 test plates were transferred 2 μl of ciprofloxacin and 10 μl of DG-5, and also to the wells with no compound (GC and SC wells) was added with 2 μl of 100% DMSO or 10 μl of DG-5 solvent. To the wells of A1-A12 and B1-B11 of the assay plate was added 98 μl of the bacterial inoculums, while to the C1-C12 and D1-D11 wells of the assay plate was added 90 μl of the bacterial inoculum. To the B12 well of the test plate was added 98 μl of the medium, and to the D12 well of the test plate was added 90 μl of the medium. After the addition process, the 5 test plates were placed with a sterile cover, centrifuged at 1000 rpm for 30 seconds, and then placed in a regular incubator at 35±2° C. for 16-20 hours.

Colony Count:

The inoculated bacteria were diluted from 10⁻¹ to 10⁻⁷ with liquid medium (e.g., 100 μl of bacterial inoculum+900 μl of 1.1×CAMHB). The above bacterial diluent (100 μl) was evenly spread on TSA plates with each dilution in duplication. After the medium was absorbed by TSA for 10 minutes, the inverted plate was incubated in an incubator at 35±2° C. for 24 hours. Bacterial inoculum usually contains 1-2×10⁸ colonies per milliliter, corresponding to 2.5-5×104 colonies per well of the test plate.

Record of Minimum Inhibitory Concentration and Colony Count:

The compound management system was opened to check the barcode and compound arrangement of each test plate. The test plate was placed on the plate reading device and the mirror was adjusted to record the bacterial growth in each well. At the same time, each test board was photographed with QCount software. The minimum inhibitory concentration of each compound was recorded by reference to the Guidelines of the Clinical and Laboratory Standards Institute. The number of colonies of the bacterial inoculum at different dilutions in the TSA plate was counted and the bacterial inoculum was calculated.

3. Results

In this study, the MICs of one test sample DG-5 and one control antibiotic ciprofloxacin against five strains of bacteria were determined by using micro-medium dilution method of the Guidelines of the CLSI. Test sample DG-5 was doubling-diluted in a 96-well plate from the highest detection concentration of 30 μg/ml. Bacterial inoculum in the assay plates were resuscitated from different solid medium plates and diluted in CAMHB or CAMHB II, while growth controls and sterile controls were placed in the assay plates. The test plates were incubated in a regular incubator at 35±2° C. for 16-20 hours, and the MIC of each compound against different bacteria was observed and recorded. The MIC values for 2 independent replicates were listed in Tables 1 and 2, respectively. The MIC of the control compound ciprofloxacin was found consistent with that reported in the literature. The bacterial inoculum size of the test plates was counted and recorded in Table 3.

TABLE 1
Minimum inhibitory concentration values of trial 1

| | Serial | Ciprofloxacin (μg/ml) | | DG-5 (μg/ml) | |
|---|---|---|---|---|---|
| Bacteria | number | Row A | Row B | Row C | Row D |
| Klebsiella | ATCC | 0.25 | 0.5 | 7.5 | 7.5 |
| pneumoniae | 700603 | | | | |
| Acinetobacter | ATCC | 16 | 16 | 3.75 | 3.75 |
| calcoaceticus | 51432 | | | | |
| Enterococcus | ATCC | >64 | >64 | 3.75 | 3.75 |
| faecalis | 700221 | | | | |
| Streptococcus | ATCC | 2 | 2 | 15 | 15 |
| pneumoniae | 49619 | | | | |
| Staphylococcus | ATCC | 0.5 | 1 | 3.75 | 3.75 |
| aureus | 43300 | | | | |

TABLE 2
Minimum inhibitory concentration values of trial 2

| | Serial | Ciprofloxacin (μg/ml) | | DG-5 (μg/ml) | |
|---|---|---|---|---|---|
| Bacteria | number | Row A | Row B | Row C | Row D |
| Klebsiella | ATCC | 0.5 | 0.25 | 7.5 | 3.75 |
| pneumoniae | 700603 | | | | |
| Acinetobacter | ATCC | 16 | 16 | 3.75 | 1.875 |
| calcoaceticus | 51432 | | | | |
| Enterococcus | ATCC | >64 | >64 | 3.75 | 3.75 |
| faecalis | 700221 | | | | |
| Streptococcus | ATCC | 2 | 2 | 15 | 15 |
| pneumoniae | 49619 | | | | |
| Staphylococcus | ATCC | 1 | 1 | 3.75 | 3.75 |
| aureus | 43300 | | | | |

TABLE 3
Number of colonies inoculated with five
strains of bacteria (trial 1 and trial 2)

| | | Trial 1 | Trial 2 |
|---|---|---|---|
| Bacteria | Serial number | (colonies/ml) | (colonies/ml) |
| Klebsiella | ATCC 700603 | 2.42E+05 | 2.37E+05 |
| pneumoniae | | | |
| Acinetobacter | ATCC 51432 | 2.36E+05 | 2.08E+05 |
| calcoaceticus | | | |
| Enterococcus | ATCC 700221 | 4.55E+04 | 5.10E+04 |
| faecalis | | | |
| Streptococcus | ATCC 49619 | 1.04E+05 | 1.18E+05 |
| pneumoniae | | | |
| Staphylococcus | ATCC 43300 | 3.25E+05 | 3.50E+05 |
| aureus | | | |

### Example 3: Determination of the Activities of the Nano-Material Composition DG-5 Against 5 Strains of Super Drug-Resistant Bacteria

The same method as in Example 2 was used to determine the activities of the nano-material composition DG-5 against 5 strains of super drug-resistant bacteria (Enterobacter cloacae, Klebsiella pneumoniae, Escherichia coli, Pseudomonas aeruginosa, and Acinetobacter baumannii). The minimum inhibitory concentration of the referenced ciprofloxacin was found consistent with the historical data. Finally, the minimum inhibitory concentration of the test sample DG-5 against 5 strains of bacteria was between 1.875-3.75 μg/ml, which was much better than the antibacterial activity of the positive control drug ciprofloxacin.

1. Material

Bacterial Strain

| | Gram staining | |
|---|---|---|
| Bacteria | classification | Serial number |
| Enterobacter | G− | EC9866 |
| cloacae | | |
| Klebsiella | G− | KP30006 |
| pneumoniae | | |
| Escherichia | G− | EC30026 |
| coli | | |
| Pseudomonas | G− | PA9347 |
| aeruginosa | | |
| Acinetobacter | G− | AB2810565 |
| baumannii | | |

Medium:

Trypticase soy agar (TSA) (BD BBL 211043), cation-adjusted Mueller Hinton broth (CAMHB) (BD BBL212322).

Reagents and Consumables:

The test sample DG-5 (300 μg/ml) was supplied by Changsha Digu Nami. Ciprofloxacin (Sigma 17850). Disposable shake flask, 250 ml (Corning 430183). Disposable plate, 100 mm (VWR 25384-302), and 96-well microtiter plate (Greiner 650162).

2. Method

Bacterial Resuscitation:

Five strains of bacteria used for the minimum inhibitory concentration test were frozen at −80° C. in a low-temperature refrigerator and resuscitated 2 days earlier. A small amount of frozen bacteria was scrapped with a sterile inoculating loop, streak-inoculated in appropriate solid medium plates, and placed in a regular incubator at 35±2° C. for 35-24 hours. 5-10 morphologically similar colonies were picked from the above culture dishes using a sterile inoculating loop, re-streaked onto a suitable solid medium plate, and was then placed in a regular incubator at 35±2° C. for 35-24 hours.

Inoculation of Bacteria:

A liquid medium was taken out of the 4° C. refrigerator and warmed up at room temperature. Five to 10 bacterial single colonies were picked from the above solid culture dish and re-suspended in 500 μl of 1.1×CAMHB, and the OD₆₀₀ was adjusted to 0.1 to 0.13 with a spectrophotometer. The bacteria were diluted 400 times with 1.1×CAMHB. The prepared bacterial inoculum was inoculated into a 96-well assay plate within 15 minutes. The number of bacteria inoculated was obtained by counting the colonies of the plates.

Preparation of Test Plate:

Test plate map (see FIG. 1): Rows A and B: ciprofloxacin (CIP), the highest test concentration being 64 μg/ml, and doubling diluted; rows C and D: DG-5, the highest test concentration being 30 μg/ml, and doubling-diluted; growth control (GC): 1.1×CAMHB or DG-5 solvent containing bacterial inoculum, no compound being present. Sterile control (SC): 1.1×CAMHB or DG-5 solvent, no compound being present.

Dilution of the Compounds:

To the starting well (A1, B1, C1 and D1) of the dilution plate was transferred 120 μl of the compound, and to other wells was transferred 60 μl of dimethyl sulfoxide (DMSO for diluting ciprofloxacin) or DG-5. Each compound was serially doubling-diluted from column 1 to column 11 (i.e., 60 μl was taken from column 1 to column 2 and mixed, and 60 μl was taken from column 2 to column 3 and mixed, and then 60 μl was taken from column 3 to column 4 and mixed, and so on, until the column 11). To the corresponding wells of each of the 5 test plates were transferred 2 μl of ciprofloxacin and 10 μl of DG-5, and also to the wells with no compound (GC and SC wells) was added with 2 μl of 100% DMSO or 10 μl of DG-5 solvent. To the wells of A1-A12 and B1-B11 of the assay plate was added 98 μl of the bacterial inoculums, while to the C1-C12 and D1-D11 wells of the assay plate was added 90 μl of the bacterial inoculum. To the B12 well of the test plate was added 98 μl of the medium, and to the D12 well of the test plate was added 90 μl of the medium. After the addition process, the 5 test plates were placed with a sterile cover, centrifuged at 1000 rpm for 30 seconds, and then placed in a regular incubator at 35±2° C. for 16-20 hours.

Colony Count:

The inoculated bacteria were diluted from 10⁻¹ to 10⁻⁷ with liquid medium (e.g., 100 μl of bacterial inoculum+900 μl of 1.1×CAMHB). The above bacterial diluent (100 μl) was evenly spread on TSA plates with each dilution in duplication. After the medium was absorbed by TSA for 10 minutes, the inverted plate was incubated in an incubator at 35±2° C. for 24 hours.

Record of Minimum Inhibitory Concentration and Colony Count:

The compound management system was opened to check the barcode and compound arrangement of each test plate. The test plate was placed on the plate reading device and the mirror was adjusted to record the bacterial growth in each well. At the same time, each test board was photographed with QCount software. The minimum inhibitory concentration of each compound was recorded by reference to the Guidelines of the Clinical and Laboratory Standards Institute. The number of colonies of the bacterial inoculum at different dilutions in the TSA plate was counted and the bacterial inoculum was calculated.

3. Results

In this study, the MICs of one test sample DG-5 and one control antibiotic ciprofloxacin against five strains of bacteria were determined by using micro-medium dilution method of the Guidelines of the CLSI. Test sample DG-5 was doubling-diluted in a 96-well plate from the highest detection concentration of 30 μg/ml. The bacterial inoculum in the assay plate was resuscitated from TSA and diluted in CAMHB, while growth controls and sterile controls were placed in the assay plates. The test plates were incubated in a regular incubator at 35±2° C. for 16-20 hours, and the MIC of each compound against different bacteria was observed and recorded. The MIC values for 2 independent replicates were listed in Tables 1 and 2, respectively. The MIC of the control compound ciprofloxacin was found consistent with that reported in the literature. The bacterial inoculum size of the test plates was counted and recorded in Table 3.

TABLE 1
Minimum inhibitory concentration values of trial 1

| | Serial | Ciprofloxacin (μg/ml) | | DG-5 (μg/ml) | |
|---|---|---|---|---|---|
| Bacteria | number | Row A | Row B | Row C | Row D |
| Enterobacter | EC9866 | >64 | >64 | 3.75 | 3.75 |
| cloacae | | | | | |
| Klebsiella | KP30006 | >64 | >64 | 3.75 | 3.75 |
| pneumoniae | | | | | |
| Escherichia | EC30026 | >64 | >64 | 3.75 | 3.75 |
| coli | | | | | |
| Pseudomonas | PA9347 | 16 | 16 | 1.875 | 1.875 |
| aeruginosa | | | | | |
| Acinetobacter | AB2810565 | >64 | >64 | 1.875 | 1.875 |
| baumannii | | | | | |

TABLE 2
Minimum inhibitory concentration values of trial 2

| | Serial | Ciprofloxacin (μg/ml) | | DG-5 (μg/ml) | |
|---|---|---|---|---|---|
| Bacteria | number | Row A | Row B | Row C | Row D |
| Enterobacter | EC9866 | >64 | >64 | 3.75 | 3.75 |
| cloacae | | | | | |
| Klebsiella | KP30006 | >64 | >64 | 3.75 | 3.75 |
| pneumoniae | | | | | |
| Escherichia | EC30026 | >64 | >64 | 3.75 | 3.75 |
| coli | | | | | |
| Pseudomonas | PA9347 | 16 | 16 | 1.875 | 1.875 |
| aeruginosa | | | | | |
| Acinetobacter | AB2810565 | >64 | >64 | 1.875 | 1.875 |
| baumannii | | | | | |

TABLE 3
Number of colonies inoculated with five
strains of bacteria (trial 1 and trial 2)

| | | Trial 1 | Trial 2 |
|---|---|---|---|
| Bacteria | Serial number | (colonies/ml) | (colonies/ml) |
| Enterobacter | EC9866 | 1.80E+05 | 3.88E+05 |
| cloacae | | | |
| Klebsiella | KP30006 | 1.53E+05 | 2.45E+05 |
| pneumoniae | | | |
| Escherichia | EC30026 | 1.93E+05 | 1.78E+05 |
| coli | | | |
| Pseudomonas | PA9347 | 2.10E+05 | 2.85E+05 |
| aeruginosa | | | |
| Acinetobacter | AB2810565 | 1.85E+05 | 3.35E+05 |
| baumannii | | | |

1. A method for treatment of drug-resistant bacteria, comprising administering to a patient in need of the treatment a nano-material composition DG 5, wherein the composition comprises: spherical nano-silver powder 1-2 g/L, glucose 1-2 g/L, and water as the rest; and the spherical nano-silver powder has a particle size of ≤0.1-5 nm, and a purity of silver ≥99.99%.

2. The method of claim 1, wherein the drug-resistant bacteria are Klebsiella pneumoniae, Acinetobacter calcoaceticus, Enterococcus faecalis, Streptococcus pneumonia, or Staphylococcus aureus.

3. The method of claim 1, wherein the drug-resistant bacteria are super drug-resistant bacteria.

4. The method of claim 3, wherein the super drug-resistant bacteria are Enterobacter cloacae, Klebsiella pneumoniae, Escherichia coli, Pseudomonas aeruginosa or Acinetobacter baumannii.

5. The method of claim 1, wherein the composition is used alone or in combination with other pharmaceutical agent.

6-8. (canceled)

9. A method of manufacturing an antibacterial agent, comprising:
providing the nano-material composition DG-5 according to claim 1; and
combining the composition with a pharmaceutically acceptable carrier;
wherein the composition is present in an amount sufficient for treatment of drug-resistant bacteria.

10. The method of claim 9, wherein the carrier is a diluent, excipient, filler, binder, wetting agent, disintegrant, absorption enhancer, surfactant, adsorption carrier, and lubricant, or a combination thereof.

11. The method of claim 9, wherein the antibacterial agent is in form of injection, tablet, pill, capsule, suspension or emulsion.
